(19) United States
(12) Patent Application Publication
(10) Pub. No.: US 2025/0041011 A1
(43) Pub. Date: Feb. 6, 2025

(54) USER INTERFACES FOR SURGICAL ROBOTIC SYSTEMS

(71) Applicant: EndoQuest Robotics, Inc., Houston, TX (US)

(72) Inventors: Seoungkyou Lee, Houston, TX (US); Yangjae Kang, Houston, TX (US); Jeihan Lee, Houston, TX (US); Jaesun Lee, Seobgnam (KR); Jiwon Choi, Houston, TX (US); Dongsuk Shin, Houston, TX (US)

(73) Assignee: EndoQuest Robotics, Inc., Houston, TX (US)

(21) Appl. No.: 18/920,441

(22) Filed: Oct. 18, 2024

Related U.S. Application Data
(63) Continuation-in-part of application No. 18/122,007, filed on Mar. 15, 2023, now Pat. No. 12,138,001.
Application No. 18/122,007 is a continuation of application No. PCT/US2022/051220, filed on Nov. 29, 2022.
(60) Provisional application No. 63/284,499, filed on Nov. 30, 2021.

(51) Int. Cl. A61B 34/00; A61B 34/37

(52) U.S. Cl. CPC A61B 34/74; A61B 34/25; A61B 34/37

(57) ABSTRACT

A user input device for a patient console of a robotic surgical system includes a user-selectable control for each of a plurality of motion devices of the patient console, the plurality of motion devices including a vertical lift device configured to provide up and down motion along a vertical axis, a pitch rotation device configured to provide a pitch rotation about a pitch axis orthogonal to the vertical axis, a translation device configured to provide sliding translation along a translation axis, and a roll rotation device to rotate about a roll axis to provide a roll to an instrument controller assembly.

### CROSS-REFERENCE TO RELATED APPLICATIONS

This application is a continuation-in part of U.S. patent application Ser. No. 18/122,007 filed Mar. 15, 2023, which is a continuation of International Patent Application No. PCT/US2022/051220 filed Nov. 29, 2022, which claims priority to and the benefit of U.S. Provisional Application No. 63/284,499, filed Nov. 30, 2021. The entire contents of each of the foregoing are incorporated herein by reference in their entirety.

### COPYRIGHT NOTICE

A portion of the disclosure of this patent document contains material which is subject to copyright protection. The copyright owner has no objection to the facsimile reproduction by any one of the patent disclosures, as it appears in the U.S. Patent and Trademark Office patent files or records, but otherwise reserves all copyright rights whatsoever.

### TECHNICAL FIELD

This disclosure is directed to various aspects of surgical robots utilizing a flexible access port or steerable overtube, which are particularly suited for use in endoluminal (endolumenal) surgical procedures. More particularly, this disclosure relates to robotically assisted transoral, transesophageal, transumbilical, intragastric, transanal and transvaginal endoscopic surgical procedures, techniques, and treatments, sometimes referred to as Natural Orifice Translumenal Endoscopic Surgery (NOTES). This disclosure also relates to Single Incision Laparoscopic Surgery (SILS), Single Port Access (SPA) surgery, Natural Orifice Trans-Umbilical Surgery (NOTUS), Laparo-Endoscopic Single-site Surgery (LESS), One Port Umbilical Surgery (OPUS), Single Port Incisionless Conventional Equipment-utilizing Surgery (SPICES), and Single Access Site Surgical Endoscope (SASSE) procedures.

Additionally, this disclosure is directed to various aspects of robots designed for performing functions in confined spaces, including industrial applications. Specifically, the systems, devices and related methods of the present invention can advantageously be applied to various nonmedical fields, such as industrial robots and remotely operated vehicles, including those used in outer space or deep-sea environments, for example in oil and gas exploration. The invention is particularly advantageous to fields requiring precise control in performing complex tasks in confined and/or difficult-to-reach structures (such as within long conduits), or in situations where access requires navigation around or through existing structures, including curved structures.

### BACKGROUND OF THE INVENTION

Minimally invasive surgical procedures such as endoluminal surgery and single-site laparoscopic surgery are known in the art and provide many benefits over traditional open or multi-port laparoscopic surgical procedures. Endoluminal surgical procedures are performed endoscopically within hollow organs using typical surgical techniques, such as dissection, suturing, cutting, and stapling. These procedures may be performed trans-orally within the upper gastrointestinal (GI) tract, trans-anally within the lower GI tract, or trans-vaginally within the abdominal cavity. A significant benefit of endoluminal surgery is that no skin incision is needed to access to the surgical site within a patient's natural lumen. This can dramatically reduce patient recovery time and can improve procedural safety. Similarly, single-site or single incision laparoscopic surgical procedures are typically performed within a patient's abdominal cavity or thoracic cavity through a single incision. This can also reduce patient recovery time and trauma, as multiple incisions are avoided, providing greater flexibility in incision location.

Robotic systems are also known in the art and have been used to perform industrial tasks. Moreover, robotic surgical systems are also known in the art and have been used to perform medical and surgical procedures, such as endoluminal and single site surgical procedures. An example of such a system is disclosed, for example, in commonly assigned U.S. Patent Application Publication 2023/0285098. This flexible robotic system includes a patient cart or console with a multi-axis positioning system, and it employs a steerable overtube assembly having a plurality of working channels for introducing surgical devices to a surgical site. The overtube assembly is disclosed in commonly assigned U.S. Patent Application Publication 2023/0210618, which is also incorporated herein by reference in its entirety. Exemplary surgical devices and end effectors or tools that can be introduced to a surgical site through a working channel of the steerable overtube assembly are disclosed in commonly assigned U.S. Patent Application Publication 2023/0248419, the disclosure of which is incorporated herein by reference in its entirety.

Systems, devices and methods in accordance with the invention can incorporate or utilize aspects of devices, systems and methods disclosed in the following, each of which is incorporated herein by reference in its entirety: U.S. Patent Application Publication Nos. 2023/0285090, 2023/0363842, 2023/0210621, 2023/0248450, 2023/0285099, 2023/0248457, 2023/0363847, 2023/0255702, 2023/0355221, 2022/0354524, 2021/0275266, 2020/0397456, and/or 2024/0058079, and U.S. patents application Ser. Nos. 18/415,502, 18/535,425, 18/596,171, 63/641, 114, 63/641, 165, 63/677,557, 63/677,576, 63/677,614, 63/677,648, and/or 18/790,627.

Applicant recognizes a need in the art for improved robotic surgical systems, devices, methods, controls, and components, especially those configured for endoluminal and single-site surgery. The present disclosure provides improvements in robotic surgical systems, devices, instruments, methods, controls, components, and other accessories and ancillary components, among others, as will be appreciated.

### SUMMARY OF THE INVENTION

The purpose and advantages of the illustrated embodiments will be set forth in and apparent from the following description. Additional benefits thereof will be realized and attained through the devices, systems, methods, controls, components, instruments and other accessories and ancillary components highlighted in the written description, claims, and the appended drawings.

To achieve these and other advantages, and in accordance with the purpose of the illustrated embodiments, one aspect of the present invention relates to a user input device for a patient console of a robotic surgical system. This device includes a user-selectable control for each of a plurality of motion devices of the patient console, the plurality of motion devices including a vertical lift device configured to provide up and down motion along a vertical axis, a pitch rotation device configured to provide a pitch rotation about a pitch axis orthogonal to the vertical axis, a translation device configured to provide sliding translation along a translation axis, and a roll rotation device to rotate about a roll axis to provide a roll to an instrument controller assembly.

The plurality of motion devices can further include a yaw rotation device configured to provide a yaw rotation about the vertical axis. An angle of the translation axis and the roll axis relative to horizontal can be a function of the pitch rotation provided by the pitch rotation device. The user interface can include a digital display screen adapted and configured to display a graphic user interface, the graphic user interface configured to display graphic representations of buttons adapted and configured to control each of the plurality of motion devices. The user interface can include a button panel, including a button corresponding to each of the plurality of motion devices. The user input device can further include a button adapted and configured to selectively disengage operability of the button panel to inhibit unintentional activation of any of the plurality of motion devices. The user input device can be arranged on a distal portion of the patient console, configured to be proximal to a patient. The user input device can be arranged on a proximal portion of the patient console, configured to be distal to the patient.

In accordance with a further aspect of the invention, a user input device for a patient console of a robotic surgical system, includes a selectable control for each of a plurality of motion devices of the patient console, the plurality of motion devices including a vertical lift device configured to provide up and down motion along a vertical axis, and a pitch rotation device supported by the vertical lift device and configured to provide a pitch rotation about a pitch axis orthogonal to the vertical axis.

The plurality of motion devices can further include a translation device supported by the pitch rotation device and configured to provide sliding translation along a translation axis, and a roll rotation device supported by the translation device to roll relative to the translation device about a roll axis to provide a roll to an instrument controller assembly, wherein an angle of the translation axis and the roll axis relative to horizontal is a function of the pitch rotation provided by the pitch rotation device.

In accordance with the invention, the plurality of motion devices can further include a yaw rotation device interposed between the vertical lift device and the pitch device and configured to provide a yaw rotation about the vertical axis, wherein a direction of the translation axis and the roll axis is a function of the yaw rotation provided by the yaw rotation device.

In accordance with a further aspect of the invention, a user interface for a robotic surgical system includes a clutch button provided in connection with a robotic controller, the clutch button adapted and configured to selectively disengage at least one motion control device of the robotic controller to allow a user to manually manipulate a position of the robotic controller and a visual status indicator provided in connection with the robotic controller, adapted and configured to display an operational status of the robotic controller.

The robotic controller can be a robotic instrument controller or a robotic videoscope controller. The clutch button can be provided on a distal end portion of the robotic controller. The at least one motion control device can be a motorized actuator for effecting linear translation of the robotic controller along a longitudinal axis thereof. The visual status indicator can include a selectable color illumination source. The clutch button and the status indicator can both be integrated into a single illuminated button.

In accordance with still a further aspect of the invention, a user interface for a surgical robotic system includes an illuminated status indicator corresponding to an operational status of a motion device of a patient console.

The illuminated status indicator can be segmented, each of a plurality of segments thereof being individually controllable. The illuminated status indicator can be adapted and configured to illuminate a portion of the segments to correspond to a position of a corresponding motion device with respect to a minimum position limit and a maximum position limit. The illuminated status indicator can include a selectable color illumination source.

In accordance with the invention, the motion device can be a translation actuator of a surgical instrument controller, and the status can be a visual representation of extension position, relative to a maximum extension thereof. The motion device can be a translation device configured to provide sliding translation along a translation axis of a instrument controller assembly and the status can be a visual representation of translation position, relative to a maximum translation thereof. The translation of the instrument controller assembly translation can also indicate a translation position of a steerable overtube relative to a maximum translation thereof. The motion device can be a vertical lift device configured to provide up and down motion along a vertical axis and the status can be a visual representation of vertical position, relative to a maximum vertical position thereof. The motion device can be a pitch rotation device configured to provide a pitch rotation about a pitch axis orthogonal to the vertical axis, and the status can be a visual representation of pitch of an instrument controller assembly relative to maximum and minimum pitch thereof. The motion device can be a roll rotation device to provide a roll to an instrument controller assembly, and the status can be a visual representation of roll of the instrument controller assembly relative to maximum and minimum roll thereof. The illuminated status indicator can be elongate in form. The illuminated status indicator can be arcuate in form. A change in color of the illuminated status indicator can represent a change in state between normal operation and a warning status. A change in illumination pattern of the illuminated status indicator can represent a change in state between a static state and an operational state.

In accordance with still a further aspect of the invention, a user input device for a physician console of a robotic surgical system includes a user-selectable control for a function of the physician console.

The user input device can include a touch screen, a tactile button or a lever. The user selectable control can be a graphical representation of a button displayed in a graphical user interface on a touch screen.

The function can include one or a plurality of ergonomics settings for adjusting components of the physician console. The user-selectable control can be for position adjustments of a motion devices of the physician console. The user-selectable control can be for position adjustments of each of a plurality of motion devices of the physician console, the plurality of motion devices. Motion devices can include: a vertical lift device configured to provide up and down motion to a body of the physician console, a vertical lift device configured to provide up and down motion to an armrest of the physician console, a horizontal translation device configured to provide distal and proximal motion to a hand control device of the physician console, and/or a horizontal translation device configured to provide distal and proximal motion to a foot pedal tray of the physician console.

The function can additionally or alternatively include one or a plurality of motion scale settings for adjusting a scaling setting between hand input gestures and output instrument movements. The hand input gestures can be translational gestures. The hand input gestures can be rotational gestures. Alternatively or additionally, the function can include an emergency stop and/or an error recovery button. The function can additionally or alternatively include a braking and/or stabilizing function for securing a position of the physician console. The braking and/or stabilizing function can be triggered through a user-selectable button or touchscreen. The user-selectable control for braking and/or stabilizing can be configured to operate a motion device for applying a brake and/or stabilizer of the physician console. The user-selectable control for braking and/or stabilizing can be a manual actuator. The manual actuator can be a lever.

### BRIEF DESCRIPTION OF DRAWINGS

So that those skilled in the art to which the subject disclosure appertains will readily understand how to make and use the devices, systems and methods of the subject disclosure without undue experimentation, embodiments thereof will be described in detail herein below with reference to certain figures, wherein:

FIG. 1 is a perspective view of an embodiment of a patient console in accordance with this disclosure;

FIG. 2 is a schematic view of the embodiment of FIG. 1, showing five degrees of freedom at five joints;

FIG. 2A is a schematic view of the embodiment as shown in FIG. 2, illustrating axes of motion (e.g., A1, A2, A3, and A4);

FIG. 3 is another view of the embodiment of FIG. 2, showing the five degrees of freedom;

FIG. 4 is an elevation view of the embodiment of FIG. 1, showing in a storage position;

FIG. 5 is a perspective view of the embodiment of FIG. 1, showing the positioning components and with the base and outer housing removed;

FIG. 6 is a perspective view of another embodiment of the positioning components of a patient console and with the base and outer housing removed;

FIG. 7 shows an embodiment of a graphical user interface (GUI) for an embodiment of a user input device of the patient console providing controls for positioning components;

FIGS. 8A and 8B illustrate a control the first joint, e.g., a vertical lift as shown;

FIGS. 9A and 9B illustrate a control the second joint, e.g., a yaw rotation device as shown;

FIGS. 10A and 10B illustrate a control the third joint, e.g., a pitch rotation device as shown;

FIGS. 11A and 11B illustrate a control the fourth joint, e.g., a translation device as

shown;

FIGS. 12A and 12B illustrate a control the fifth joint, e.g., a roll rotation device;

FIG. 13 illustrates an embodiment of a robotic surgical system in accordance with this disclosure;

FIG. 14 illustrates a further embodiment of an instrument controller assembly in accordance with the invention, including various user interface features;

FIG. 15A is a front isometric view of an embodiment of a physician console (or operator console in non-medical settings) for systems in accordance with the invention;

FIG. 15B is an illustration of the armrest of the physician console of FIG. 15A, including graphical and tactile user interfaces provided for adjustment and control of the physician console;

FIG. 16A is a side view of the physician console of FIG. 15A;

FIG. 16B is a detail view of a user interface for application of a brake of the physician console of FIG. 15A; and

FIGS. 17A-C illustrate embodiments of graphical user interfaces for adjusting functions of the physician console of FIG. 15A.

### DETAILED DESCRIPTION OF THE INVENTION

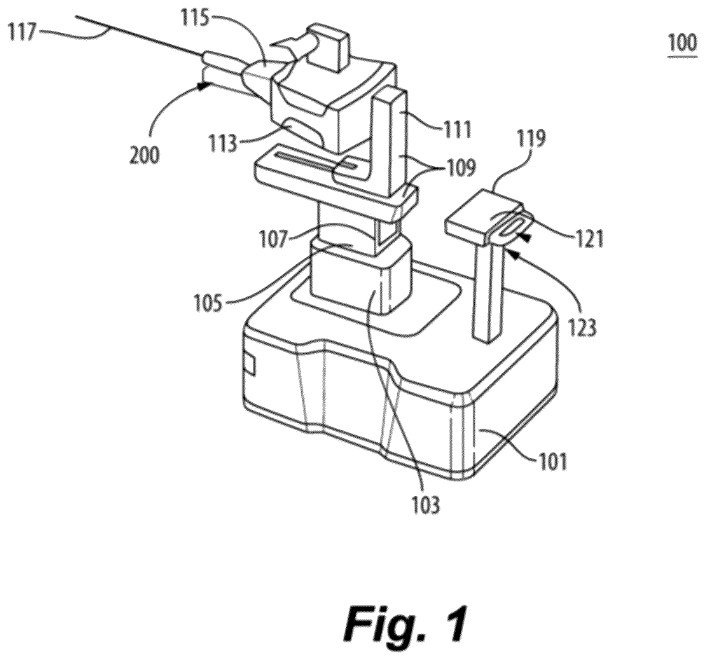

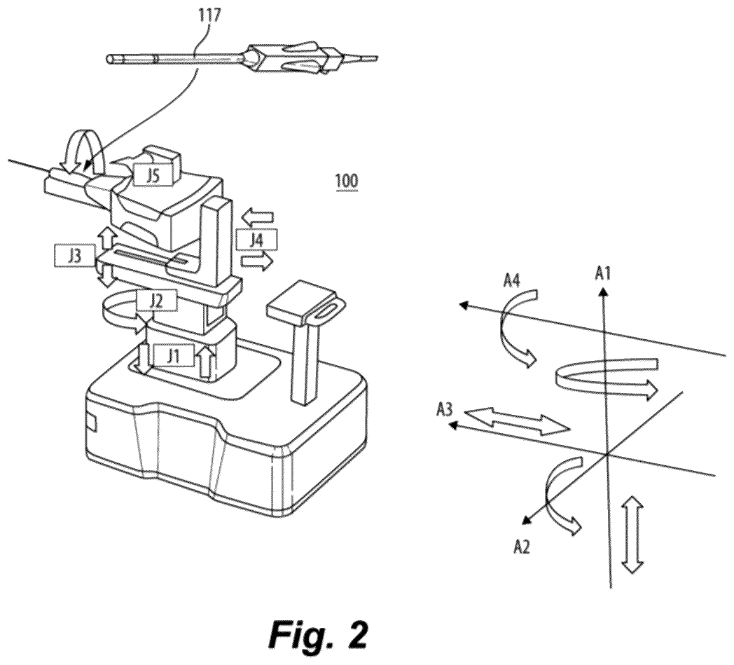

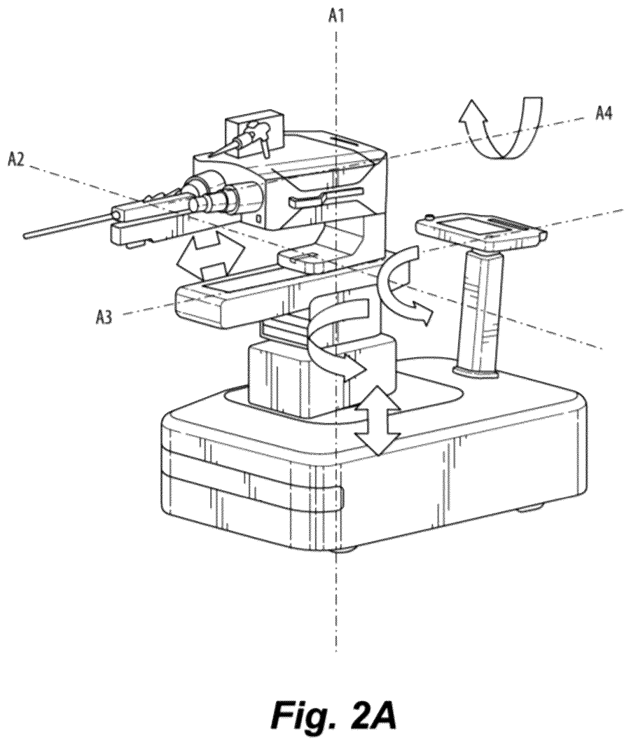

Reference will now be made to the drawings wherein like reference numerals identify similar structural features or aspects of the subject disclosure. For purposes of explanation and illustration, and not limitation, an illustrative view of an embodiment of a system in accordance with the disclosure is shown in FIG. 1 and is designated generally by reference character 100. Other embodiments and/or aspects of this disclosure are shown in FIGS. 2-13.

Referring to FIGS. 1-6 a patient console 100 (e.g., a cart) for a robotic surgical system can include a base 101 and a positioning arm 102. The positioning arm 102 can include a vertical lift 103 (e.g., joint J1) attached to a top of the base 101 and configured to provide up and down motion in a vertical axis A1. The patient console 100 can include a yaw rotation device 105 (e.g., joint J2) attached to the top of the vertical lift 103 and configured to provide a yaw rotation about the vertical axis A1. The patient console 100 can include a pitch rotation device 107 (e.g., joint J3) attached to the top of the yaw rotation device 105 to provide a pitch rotation about a pitch axis A2 orthogonal to the vertical axis A1. The patient console 100 can include a translation device 109 (e.g., joint J4) attached to the top of the pitch rotation device 107 and configured to provide sliding translation along a translation axis A3. The patient console 100 can include a roll rotation device 111 (e.g., joint J5) attached to the translation device 109 to roll relative to the translation device 109 about a roll axis A4 to provide a roll to an instrument controller assembly 113.

An angle of the translation axis A3 and the roll axis A4 relative to horizontal can be a function of the pitch rotation provided by the pitch rotation device 107. A direction of the translation axis A3 and the roll axis A4 can be a function of the yaw rotation provided by the yaw rotation device 105.

The console 100 can include an instrument controller assembly 113 connected to the roll rotation device 111, the instrument controller assembly 113 including one or more instrument controllers 115 for controlling a medical device (not shown) for performing a surgical operation. The vertical lift 103, the yaw rotation device 105, the pitch rotation device 107, the translation device 109, and the roll rotation device 111 can provide 5-degrees of freedom to the instrument controller assembly 113 (and thus the overtube 117 mounted thereon), for example. In certain embodiments, the base 101 can be configured to move relative to a floor to provide an additional degree of freedom of motion.

The patient console 100 can be configured to allow for positioning of a medical device for a transanal procedure, a transoral procedure, or a transvaginal procedure. Any other suitable procedure is contemplated herein.

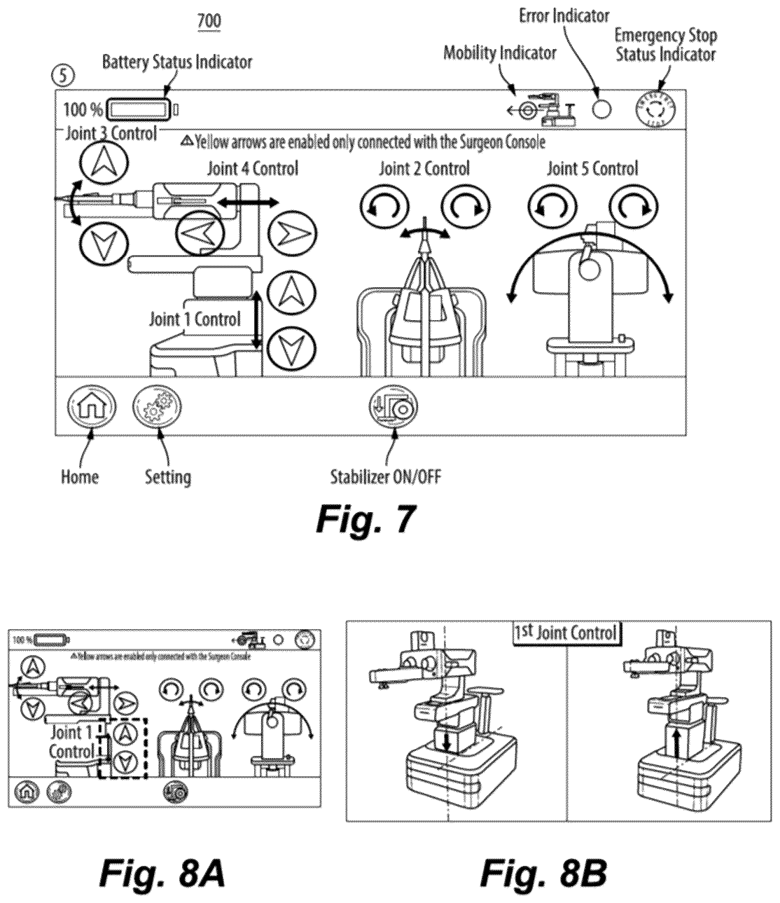

The system 100 can include a user input device 119 attached to the base 101 and configured to control each of the vertical lift 103, the yaw rotation device 105, the pitch rotation device 107, the translation device 109, and the roll rotation device 111. Referring additionally to FIG. 7, the user input device 119 can include a display 121 (e.g., a touchscreen) having a graphical user interface (GUI) 700 for controlling each of the vertical lift 103, the yaw rotation device 105, the pitch rotation device 107, the translation device 109, and the roll rotation device 111. As shown, the GUI 700 can be configured to indicate an orientation and position of the instrument controller assembly 113 from one or more angles. The GUI 700 can be configured to have any suitable digital buttons, inputs, indicators, images, text, and/or other content (e.g., arrow buttons indicating the direction of rotation as shown and/or any other indicators and/or control buttons.

In certain embodiments, one or more of the vertical lift 103, the yaw rotation device 105, the pitch rotation device 107, the translation device 109, and the roll rotation device 111 can be controlled by a remote surgeon console in addition to the user input device 119, for example. In certain embodiments, the user input device 119 can control all of the vertical lift 103, the yaw rotation device 105, the pitch rotation device 107, the translation device 109, and the roll rotation device 111, and a remote surgeon console can control the translation device 109 and the roll rotation device 111 only.

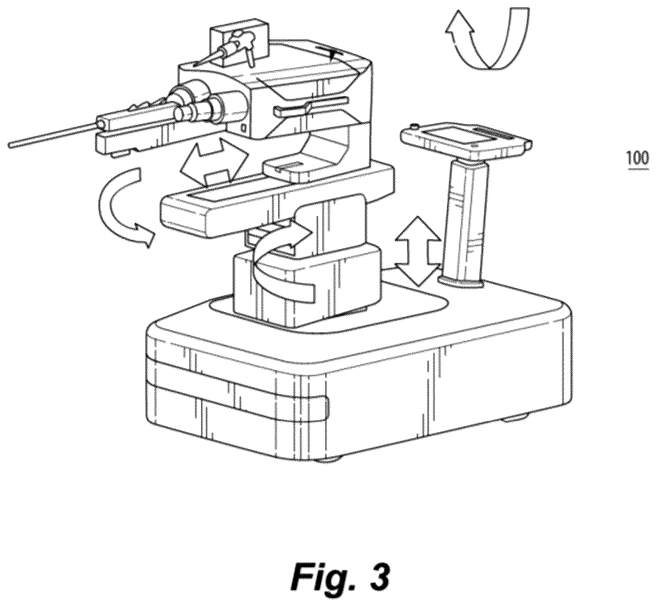

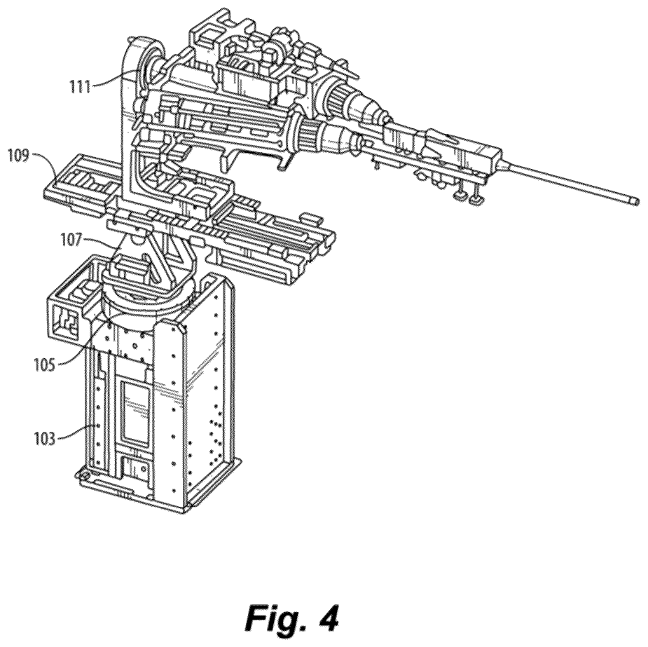

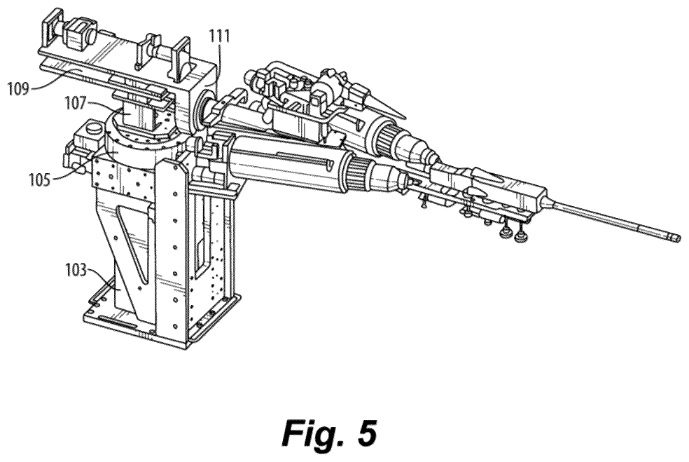

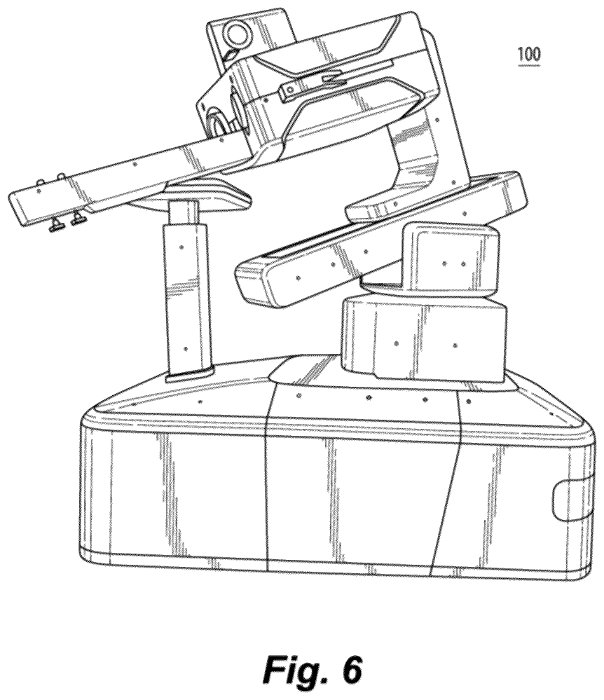

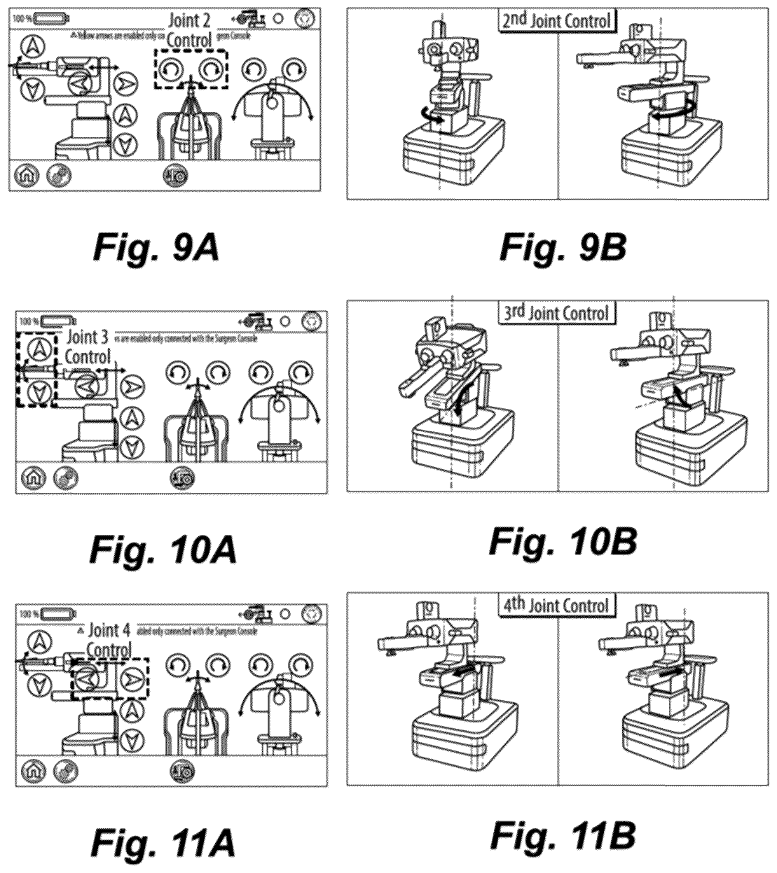

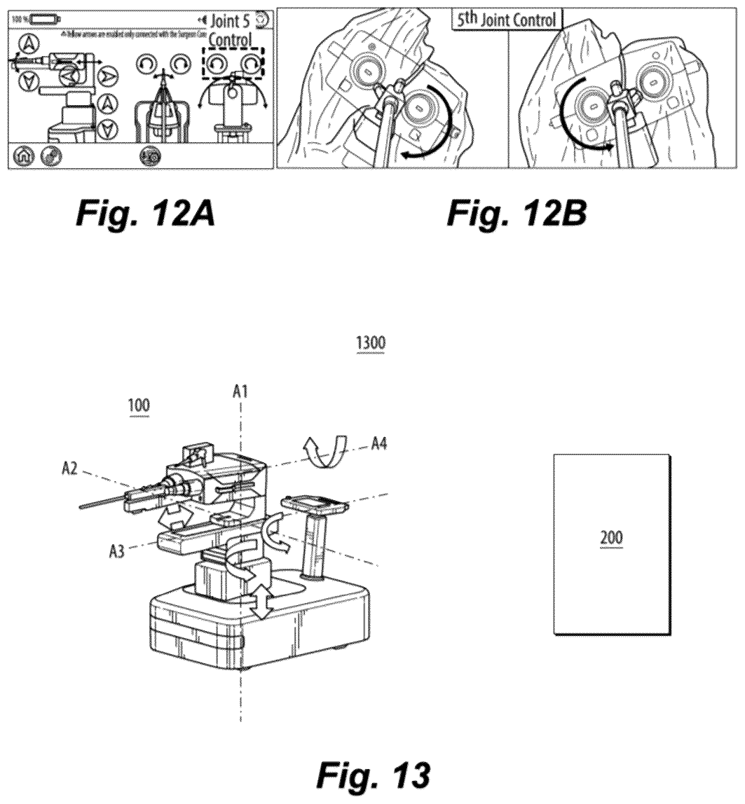

FIG. 1 is a perspective view of an embodiment of a patient console in accordance with this disclosure. FIG. 2 is a schematic view of the embodiment of FIG. 1, showing five degrees of freedom at five joints (e.g., J1, J2, J3, J4, J5). FIG. 3 is another view of the embodiment of FIG. 2, showing the five degrees of freedom. FIG. 4 is an elevation view of the embodiment of FIG. 1, showing in a storage position. FIG. 5 is a perspective view of the embodiment of FIG. 1, showing the positioning components and with the base and outer housing removed. FIG. 6 is a perspective view of another embodiment of the positioning components of a patient console and with the base and outer housing removed. FIG. 7 shows an embodiment of a graphical user interface (GUI) for an embodiment of a user input device of the patient console. FIGS. 8A and 8B illustrate a control the first joint (e.g., J1 as shown in FIG. 2), e.g., a vertical lift as shown. FIGS. 9A and 9B illustrate a control the second joint (e.g., J2 as shown in FIG. 2), e.g., a yaw rotation device as shown. FIGS. 10A and 10B illustrate a control the third joint (e.g., J3 as shown in FIG. 2), e.g., a pitch rotation device as shown. FIGS. 11A and 11B illustrate a control the fourth joint (e.g., J4 as shown in FIG. 2), e.g., a translation device as shown. FIGS. 12A and 12B illustrate a control the fifth joint (e.g., J5 as shown in FIG. 2), e.g., a roll rotation device.

Embodiments can be used for robotic surgical systems, for example. Any suitable uses and/or embodiments for use are contemplated herein.

Embodiments include five degree of freedom positioning patient console 100 (e.g., a patient cart). Embodiments can include an advantageous order of stacking of the degrees of freedom.

Embodiments can provide movements for the position of the overtube 117 attached to the overtube arm 200. Embodiments can include five joints and each joint provides specific motion. In certain embodiments, joint 1 (e.g., J1 as shown in FIG. 2) can provide up/down motion within 0-400 mm. In certain embodiments, joint 2 (e.g., J2 as shown in FIG. 2) can provide rotation motion within −90-+150 degrees. In certain embodiments, joint 3 (e.g., J3 as shown in FIG. 2) can provide tilting motion within 0-35 degrees downwards. In certain embodiments, joint 4 (e.g., J4 as shown in FIG. 2) can provide translation motion within 0-400 mm. In certain embodiments, joint 5 (e.g., J5 as shown in FIG. 2) can provide rolling motion within −170-+170 degrees.

In certain embodiments, all of the joints can be controlled by a nurse using the touchscreen on the patient console 100. Joint 4 and Joint 5 can be controlled by a surgeon by using the hand control devices and overtube pedal on the surgeon console (not shown).

Certain embodiments can include an interface (e.g. user input device 119) that has a touchscreen (e.g. the display 121), a base cart handle 123, and a stabilizer (not shown). Certain embodiments can allow control of the mobility of the patient console 100 system with drive control switches and the direction of force applied on the base cart handle 123. Certain embodiments can allow control via the base cart handle 123 is activated only when the drive control switches are pressed down halfway and held. Certain embodiments can be immobilized by activating the stabilizers via a touchscreen to prevent unwanted movement during surgery.

Embodiments of a user input device 119 can be used to control the positioning arm 102 and patient console 100. Embodiments of a user input device can indicate the status of the patient console 100. Embodiments of a user input device 119 can provide a setting menu for the base cart and its touchscreen. Embodiments of a user input device 119 can display buttons to control movements of the positioning arm 102.

Embodiments of a touchscreen GUI 700 can include a loading user interface that displays immediately when the power is on and initializes the patient console 100 and touchscreen. The GUI can include a home screen that provides access menu buttons to the Pose Setting, Patient Cart, and Diagnosis, for example. Embodiments of a GUI 700 can include a Patient Cart Setting Screen that provides detailed settings of the patient console 100 to adjust values related to mobility. The GUI 700 can include a Setting Screen that provides a setting menu to adjust the brightness of the touch screen. The GUI 700 can include a pose setting screen that provides touch buttons to adjust the pose setting of each joint of the positioning arm 102. In FIG. 7, various modes of exemplary indicators are provided in the GUI 700. Embodiments of a GUI can include a mobility indicator that shows whether the patient console 100 can be moved or not. Embodiments can include an error indicator that shows normal or abnormal status of the system with two colors of green and orange. If the error is occurred, orange color can be showing.

An emergency stop indicator can show activation of the emergency stop. When the emergency stop is activated, the icon can be changed to orange and red colors. Embodiments can include a home button that can provides function to return to the home screen. Embodiments can include a setting button that provides a function to activate a setting menu. Embodiments can include a stabilizer on/off button that provides a function to activate/deactivate the stabilizer of the patient console 100.

In certain embodiments, a user can move the positioning arm 102 to the required target region by using the positioning arm 102 touchscreen controls. A user can insert the overtube 117 into the patient, and then move the positioning arm 102 to align with the overtube 117. A user can then connect the overtube 117 to the overtube arm 200 and tighten the knobs.

The user can control the positioning arm 102 using the touchscreen controls provided on the patient console 100. The user can push the buttons provided on the touchscreen to move each joint of the positioning arm 102. The positioning arm 102 can provide five degrees of freedom of motion. A user can press and hold the button shown on the touchscreen to move the joint in the specified direction. The user can release the touchscreen button to stop moving the positioning arm 102. Table 1 shows an embodiment of motions provided by each joint J1-J5.

TABLE 1

| Joint | Motion Direction | Motion Direction |
|---|---|---|
| J1 | Up | Down |
| J2 | Right | Left |
| J3 | Head Up | Head Down |
| J4 | Backward | Forward |
| J5 | Counterclockwise | Clockwise |

In certain embodiments, a surgeon can control the Joints 4 (e.g., J4) and 5 (e.g., J5) of the positioning arm 102 using hand control device along with an overtube pedal on a surgeon console. Any suitable other control scheme is contemplated herein.

In accordance with at least one aspect of this disclosure, referring to FIG. 13, a robotic surgical system 1300 can include a patient console 100. The patient console 100 can be any patient console disclosed herein, e.g., as described above. The robotic surgical system 1300 can include a surgeon console 200 configured to allow a surgeon to remotely operate one or more controllers connected to the patient console 100 (e.g., wirelessly or via a wired connection) to perform robotic surgery or other suitable medical procedure. Any suitable surgeon console 200 and/or control inputs are contemplated herein. In certain embodiments, the surgeon console 200 can be configured to allow control of one or more of the vertical lift 103, the yaw rotation device 105, the pitch rotation device 107, the translation device 109, and the roll rotation device 111 for positioning the medical instrument attached to the patient console 100 in situ. For example, the roll rotation device 111 can be configured to be operated by one or more hand controls of the surgeon console 200 to allow rotation of the overtube 117 (which contains one or more medical instruments and/or camera) about axis A4 to rotate the distal end of the medical device(s), camera, and/or overtube 117 in situ.

In accordance with at least one aspect of this disclosure, a method for performing a robotic medical procedure can include using a vertical lift 103 attached to a top of a base 101 of a patient console to provide up and down motion in a vertical axis A1, using a yaw rotation device 105 attached to the top of the vertical lift 103 to provide a yaw rotation about the vertical axis A1, using a pitch rotation device 107 attached to the top of the yaw rotation device 105 and configured to provide a pitch rotation about a pitch axis A2 orthogonal to the vertical axis A1, using a translation device 109 attached to the top of the pitch rotation device 107 and configured to provide sliding translation along a translation axis A3, and using a roll rotation device 111 attached to the translation device 109 to roll relative to the translation device 109 about a roll axis A4 to provide a roll to an instrument controller assembly 113. An angle of the translation axis A3 and the roll axis A4 relative to horizontal can be a function of the pitch rotation provided by the pitch rotation device 107 (about axis A2). A direction of the translation axis A3 and the roll axis A4 can be a function of the yaw rotation about the vertical axis A1 provided by the yaw rotation device 105.

The method can include using an instrument controller assembly 113 having one or more instrument controllers 115 connected to the roll rotation device 111 for controlling a medical device for performing a surgical operation, and using the vertical lift 103, the yaw rotation device 105, the pitch rotation device 107, the translation device 109, and the roll rotation device 111 provide 5-degrees of freedom to the instrument controller assembly 113. The method can include moving the base 101 of the patient console 100 relative to a floor (on which the patient console 100 is standing) to provide an additional degree of freedom of motion. The method can include positioning a medical device for a transanal procedure, a transoral procedure, or a transvaginal procedure.

The method can include controlling each of the vertical lift 103, the yaw rotation device 105, the pitch rotation device 107, the translation device 109, and the roll rotation device 111 with a user input device. The method can include using a graphical user interface (GUI) to control each of the vertical lift 103, the yaw rotation device 105, the pitch rotation device 107, the translation device 109, and the roll rotation device 111. The method can include any other suitable method(s) and/or portion(s) thereof.

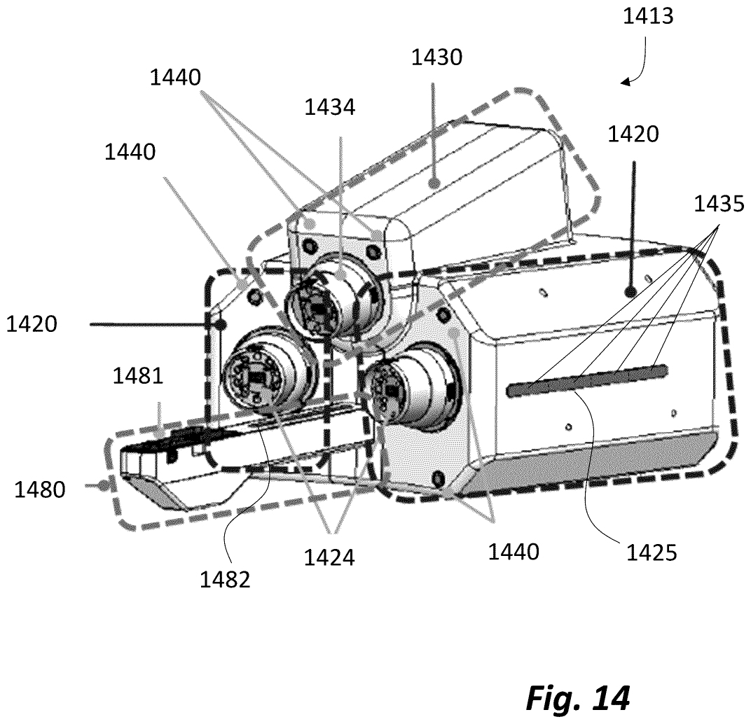

With reference to FIG. 14, a further embodiment of an instrument controller assembly 1413 in accordance with the invention is illustrated, including various user interface features. The instrument controller assembly 1413 includes instrument controllers 1420, equipped with sanitary drape adapters 1424, a videoscope controller 1430 with a corresponding drape adapter 1434. The drape adapters 1424, 1434 are detachable from their respective controllers to facilitate sterilization and the placement for draping material over the instrument controller assembly 1413. As in the above-described embodiment, an overtube arm 1480 extends distally from the instrument controller assembly 1413 and provides a point to attach the overtube (e.g., 117), and enables control of its robotic steering functions. The robotic steering functions are accomplished through actuators housed in the overtube arm 1480. The overtube arm 1480 is also shown with a sterile drape adapter plate 1481, functioning to maintain a sterile field, similarly to instrument drape adapters 1424 and the videoscope drape adapter1434. In one embodiment, the overtube arm 1480 is axially static relative to the instrument controller assembly 1413. Longitudinal translation thereof can therefore be accomplished by translational movement of the instrument controller assembly 1413.

User interface features in connection with the overtube arm 1480 include an elongate visual indicator 1482, which, in accordance with a preferred aspect, is illuminated. The visual indicator 1482, as with other indicators described herein, can incorporate any controllable display technology, including LED, OLED, electroluminescent materials, backlit LCD, and the like. In certain embodiments, the visual indicator 1482 comprises a linear array of LED modules. In certain embodiments, the LED modules are capable of displaying a variety of colors, such as through RGB or RGBW modules.

In connection with visual indicators described herein, color changes or illumination patterns (e.g. solid, blinking, sequential illumination, partial illumination) can be selected so as to guide a user through setup steps, indicate the position of components, signal active motion of a corresponding component, or indicate a fault or system error related to the corresponding component or other system component(s).

In guided system setup, a blinking pattern can draw a user's attention to the next prescribed setup step, as determined by the system. For example, upon initial preparation of a surgical robotic system, draping is required to maintain a sterile field in the operating suite. Accordingly, the systems can be equipped with programming and sensors to both guide a setup process and detect when each step is completed. For example, a blinking status indicator can guide the draping of the next portion of the surgical robot. Once the sensor detects that the step is complete (or the operator or technician manually confirms that the step is complete, such as through a menu), the status indicator's color and/or illumination pattern can change, such as from blinking to solid illumination, or from blue to green. A visual indicator for a subsequent setup step can then be illuminated, and the process continues.

As will be described in further detail below, in certain embodiments, the visual indicator 1482 and other visual indicators disclosed herein can be configured to illuminate so as to draw the attention of a user during a setup process and/or indicate that a component, such as a linear translation stage supporting the instrument controller assembly 1413 and/or the overtube itself, is actively moving. The visual indicators, such as visual indicator 1482, can be configured to display a system status and/or the extent of travel of a component, such as of the linear translation stage and/or the overtube, by partially illuminating along its length (e.g. as a scale or bar graph). Various segments can be provided and can be illuminated independently. The number of segments may be any number suitable to perform the prescribed function thereof.

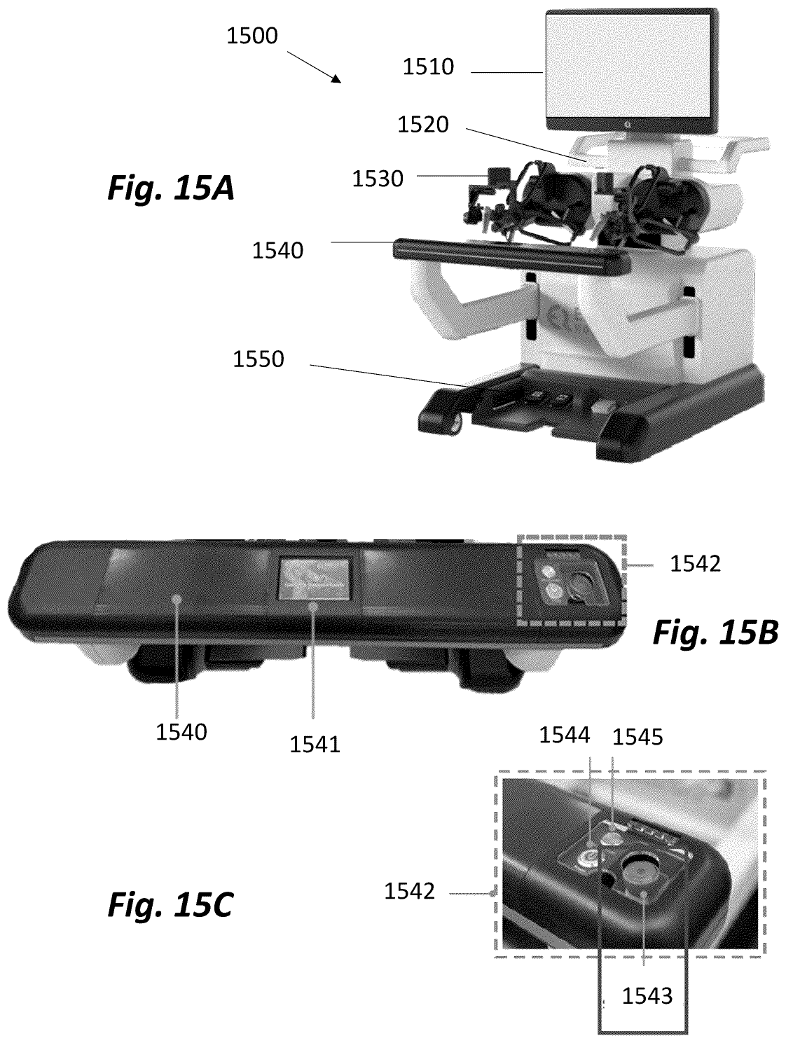

The overtube arm 200, 1480, along with its corresponding robotic controller, serves as the attachment interface and driver of flexural motion of the overtube 117 at the surgical target, as controlled by the user at the physician console (e.g. 200, 1500 in FIG. 15A). The overtube 117 flexural motion at the surgical target is controlled by the operator at the physician console (e.g. 200, 1500) with two degrees of freedom (left/right and up/down) via the robotic overtube controller housed within the overtube arm 200, 1480. Once the overtube 117 is docked to the overtube controller, LEDs of the visual indicator 1482 illuminate to confirm the connection status. This is achieved by aligning and pressing the overtube hub (the proximal control end and connection interface) into connection with the overtube controller, such as via the overtube drape adapter 1481. When the overtube 117 engages with the controller of the overtube arm 200, 1480, the user will observe the visual indicator 1482 on the overtube arm 1480 change to a green color for confirmation of complete connection, for example.

In further aspects of the invention, the systems can be configured to illuminate the visual indicator 1482 in various colors and patterns to convey various status messages visually to an operator or user. The following are exemplary embodiments: In one embodiment, the visual indicator 1482 in a solid white state indicates a docking state and/or a ready state for draping. In another embodiment, the visual indicator 1482 in a solid blue state indicates that the sanitary drape adapter has been attached, but the overtube has not yet been attached. In one embodiment, the visual indicator 1482 in a solid yellow state indicates the sanitary drape adapter and the overtube have been attached. In another embodiment, the visual indicator 1482 in a blinking white state indicates that a self-test is in progress and/or that the robotic controller is performing an engagement or alignment process with the overtube. In one embodiment, the visual indicator 1482 in a solid green state indicates that the overtube is fully engaged with its controller and ready to operate. In one embodiment, the visual indicator 1482 in a blinking green state indicates that the overtube is under active control by the physician console (e.g. 200, 1500). In one embodiment, the visual indicator 1482 in an off state indicates the patient console (e.g., 100) is off, or alternatively that there is an error, or an emergency state has been detected or initiated by an operator.

Similarly, the instrument controller assembly 1413 can also include one or more visual indicators 1425 provided on the side or sides of the instrument controller assembly 1413. These indicators can display the status of the instrument controllers 1420 or other system messages. One or more visual indicators can also be provided corresponding to the videoscope controller 1430. These visual indicators 1425 are elongate in form and can be configured to represent the status or position of a corresponding instrument controller 1420, and/or optionally of the videoscope controller 1430. Each of the visual indicators 1425 can be configured to display the extent of travel of a corresponding instrument by partially illuminating along its length (e.g. as a scale or bar graph). Various segments can be provided and can be illuminated independently. Four segments 1435 are shown for illustrative purposes only and segment number may be any suitable to perform the prescribed function thereof.

As with the visual indicator 1482 on the overtube arm 1480, the visual indicator 1425 on the instrument controller assembly 1413 can be configured to illuminate to draw a user's attention during a setup process, and/or indicate that a component (such as the corresponding instrument controller and the instrument) is actively moving, and/or display a system status information, and/or to display the extent of travel of a component (such as linear translation of a corresponding instrument controller) by partially illuminating along its length. Suitable illumination technologies can include those described above in connection with the visual indicator 1482.

In further aspects of the invention, the subject systems can be configured to illuminate the visual indicator 1425 in various colors and patterns to visually convey different status messages to an operator or user. The following are exemplary embodiments: In one embodiment, the visual indicator 1425 in a blinking blue state indicates that the patient console is initiating operation (i.e. starting up). In another embodiment, the visual indicator 1425 in a blinking white state indicates that a self-test is in progress and/or that the robotic controller is performing an engagement or alignment process with the overtube 117. In one embodiment, the visual indicator 1425 in a solid green state indicates that the overtube 117 is fully engaged with its controller and ready to operate. In one embodiment, the visual indicator 1425 in a blinking green state indicates the overtube 117, an instrument and/or other system component is under active control by the physician console (e.g. 200, 1500). In one embodiment, the visual indicator 1425 in a solid blue state indicates a cleaning state. In one embodiment, the visual indicator 1425 in a solid yellow state indicates that a recoverable fault has been detected, or that a manual emergency stop button has been activated and that the operator can recover system control through system functions such as a recovery button on the physician console. In one embodiment, the visual indicator 1425 in a solid red state indicates a non recoverable fault of the instrument controller or other system component has been detected. In such an instance, a restart of the patient console may be required and can also be indicated.

Additionally, the user interface features of instrument controller assembly 1413 further include one or more clutch buttons 1440. The clutch buttons 1440 are adapted and configured to disengage at least one motion control device of a corresponding robotic instrument controller 1420 or videoscope controller 1430, allowing a user to manually manipulate a position of the controller. In certain embodiments, more than one (e.g. two) clutch buttons 1440 can be provided at a controller 1420 or 1430 to facilitate case of use by an operator. In certain embodiments, each clutch button 1440 is adapted and configured to disengage a drive assembly for the axial translation of a respective controller, enabling manual movement of a distal portion for connecting and disconnecting instruments and/or the videoscope.

The clutch buttons 1440, or the instrument to which they correspond, can be provided with one or more visual indicators, such as illuminated indicators, which can include the above-described illumination types. In one alternative embodiment, the visual indicators associated with the clutch buttons 1440 or the instruments they control (e.g., the instrument controllers 1420 or videoscope controller 1430), can include one or more digital displays to show detailed or dynamic information or symbols. Any such visual indicators can be integrated into the clutch button interface itself, for example, by an illuminated button, or a transparent or clear button, or touch-sensitive surface over a digital display. In the illustrated embodiment, the functions are integrated into an illuminated button which can display various colors depending on a status of the corresponding device.

In further aspects of the invention, the systems can be configured to illuminate the visual indicators associated with instrument controller 1420, videoscope controller 1430, and/or the clutch buttons 1440 in various colors and patterns to visually convey different status messages to an operator or user. The following are exemplary embodiments: In one embodiment, a blinking white state indicates a self-test is in progress. In one embodiment, a solid white state indicates a loading/unloading position. In one embodiment, a blinking blue state indicates a drape has not yet been attached. In one embodiment, a solid blue state indicates a drape is attached, but an instrument or videoscope is not yet attached. In one embodiment, a solid yellow state during a setup procedure indicates that an instrument or videoscope needs to be retracted to a loading position before starting or completing a self test. In one embodiment, a solid yellow state during regular operation indicates that an instrument or videoscope is attached but not in a ready position, requiring manual advancement. In one embodiment, a solid green state indicates that an instrument or videoscope is in a ready position. In one embodiment, if no indicator light is present during a setup process, it indicates that draping is in process, but not all drape components have been attached. In one embodiment, if no indicator light is present during normal operation, it signals an error or emergency state, or that a recoverable fault has been detected.

In a further embodiment, a blinking red state indicates a warning, such as an instrument error. In one embodiment, a solid white state signals an instruction to an operator or technician to detach a corresponding instrument or videoscope. In one embodiment, a blinking white state signals an instruction to an operator or technician to attach a corresponding instrument or videoscope. In one embodiment, a solid yellow state warns the operator or technician to take caution, indicating that an instrument is not ready to operate. In one embodiment, a blinking yellow state indicates that a homing function is being performed. In one embodiment, a solid cyan state indicates that an instrument or videoscope should be unlocked by an operator or technician. In one embodiment, a blinking cyan state indicates that an instrument or videoscope has been successfully detected. In one embodiment, a solid green state indicates that an instrument or videoscope is ready to operate. In one embodiment, a blinking green state indicates that an instrument or videoscope is actively operating. In one embodiment, a solid blue state indicates that an instrument or videoscope can be moved manually.

In further embodiments, when a digital display is used as a status indicator for each instrument controller 1420 and/or the videoscope controller 1430, more detailed status indications are possible, including text prompts that provide a message or instructions to an operator or technician. In such embodiments, a house icon can indicate that a homing function is being performed. The house icon can appear in any colors, such as green to indicate normal operation or yellow to indicate a delay in operation due to the process. Icons of any color or shape can be provided, such as red to indicate an error or a stopped function. Further graphical animations can be used, such as a moving circular animation to indicate an ongoing process such as instrument identification. A single or double-headed arrow can be displayed to indicate that an instrument or videoscope controller can be operated manually. Such an arrow can be animated or static in any colors such as blue or green to indicate that the process may proceed. In some embodiments, gray icons can instruct an operator to attach or detach an instrument or videoscope. In one embodiment, a solid icon can indicate instructions to detach an instrument. In one embodiment, a blinking icon can indicate to attach an instrument. In one embodiment, a solid cyan icon indicates to unlock an instrument. In one embodiment, a blinking cyan icon indicates that instrument detection is in progress. In one embodiment, a solid yellow icon indicates that an instrument is ready to operate. In one embodiment, a blinking yellow icon indicates that an instrument or videoscope is not ready to operate. In one embodiment, an animated yellow icon indicates that a valid instrument has been detected. In one embodiment, an animated red icon indicates that an invalid instrument has been detected, such as an “X” icon. In one embodiment, a solid green icon indicates that an instrument or videoscope is actively moving. These foregoing status conditions can also be reflected in other user interfaces, including position indicators, system GUIs, and the like.

Example 1—Use of Clutch Button and Visual Indicator During Draping: The following example demonstrates the use of a clutch button 1440 and visual user interface feedback during the process of installing the videoscope drape adapter 1434 or instrument drape adapter 1424, as performed by an operator or technician: 1) Extend the videoscope 1430 or instrument controller 1420 by pressing and holding a corresponding clutch button 1440, and advancing the controller 1430 until the visual indicator (e.g. clutch button 1440 LED) stops blinking blue. 2) Install the sterile drape and drape adapter 1424, 1434, if applicable. 3) Retract the videoscope controller 1430 by pressing and holding a clutch button 1440 corresponding to the videoscope controller 1430 until the LED changes to a solid blue color, manually retracting the videoscope controller 1430 back to the loading position, and then releasing the corresponding videoscope controller clutch button 1440.

Example 2—Advancing Videoscope Controller to Ready Position: To advance the videoscope controller 1430 to a ready position, the following steps are taken: 1. Press and hold videoscope controller 1430 clutch button 1440 and manually advance videoscope controller 1430 (towards the patient) until it reaches a flush position (e.g. aligned with a distal end of the overtube). 2. Release the videoscope controller 1430 clutch button 1440 when the flush position is reached. The physician may also advance the videoscope 1430 to the ready position using the physician console (e.g., 200, 1500). 3. If necessary, press and hold videoscope controller 1430 clutch button 1440 and manually advance videoscope controller 1430 to the ready position in communication with the physician. Observe the visual indicator (such as LED) on the respective videoscope controller 1430 change to a green color for confirmation of the ready status.

Example 3—Detaching and removing an instrument: The instruments are removed from the instrument controllers 1420 one at a time. To remove an instrument for exchange, inspection, and/or cleaning the end effector with sterile gauze, or final removal after the procedure is completed, the following steps are taken for each instrument, as needed: 1. On the respective instrument controller (left or right) 1420, press and hold a corresponding clutch button 1440 and manually retract the instrument controller 1420 (away from the patient) to the loading position, indicated by the visual indicator (e.g., an LED changing to a solid white color). 2. Release the instrument controller 1420 clutch button 1440 to maintain the instrument controller 1420 in the loading position. 3. This example can further include the following step: Wait for the instrument to auto-align to its removal position (the instrument controller 1420 will automatically rotate until the red latch on the instrument drape adapter (e.g. 1424) is at “12 o'clock”.

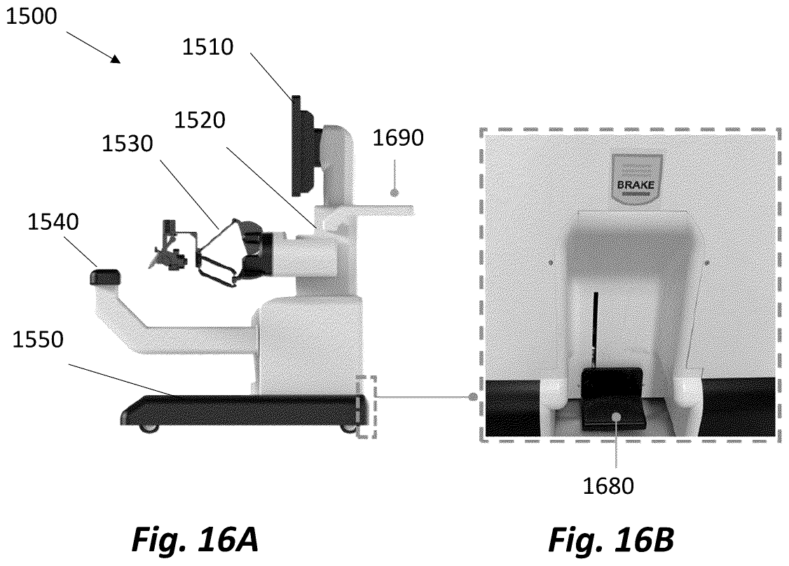

Turning now to FIGS. 15A-15C and 16A-16B, an embodiment of a physician console 1500 (or operator console in nonmedical settings) for systems in accordance with the invention is illustrated. The physician console 1500 includes a display 1510 for displaying system images and functions to the physician, a main body 1520 for supporting the display 1500 and hand control devices 1530, which the physician uses to control connected instruments (e.g., the videoscope and overtube, depending on the specific implementation). The physician console 1500 also includes an armrest 1540 and a foot tray 1550 which includes foot pedals for controlling device and system functions, such as electro-surgical instrument functions.

As illustrated in FIG. 15B, a touchscreen user interface 1541 can be provided in the armrest 1540 for displaying a GUI for configuring adjustments and scale functions of the physician console 1500. Additionally, a tactile user interface 1542 can be provided for various functions. As best illustrated in FIG. 15C, the tactile interface 1542 can include an emergency stop button 1543, a power button 1544 and a recovery button 1545. The emergency stop button 1543 can be used to stop all movements of the patient console 100 and physician console 1500 and prevent further movement until the system is restored, for example, by using the recovery button 1545. The recovery button 1545 can also be used resume system control after fixing any minor component fault detected by the system.

As can be seen in the side view of FIG. 16A, the physician console 1500 can include a user interface handle 1690 for transporting and positioning the physician console 1500 by an operator, technician, or others. Also seen in FIG. 16A, as well as in FIG. 16B, is a position of a brake and/or stabilizer user interface 1680 adapted and configured to brake and/or stabilize the physician console 1500. Such user interface can be provided to actuate a brake and/or stabilizer of the physician console 1500. The brake and/or stabilizer user interface can be provided in a graphical interface, as a button, as a lever, or as a pedal. In the illustrated embodiment, the brake actuator user interface is a pedal. Such user interface can also be provided for the patient console 100, as seen in the graphical user interface shown in FIG. 7, where an actuator drives movement of the stabilizer, for example. Alternatively, a manually-applied brake or stabilizer can be used in connection with the patient console 100.

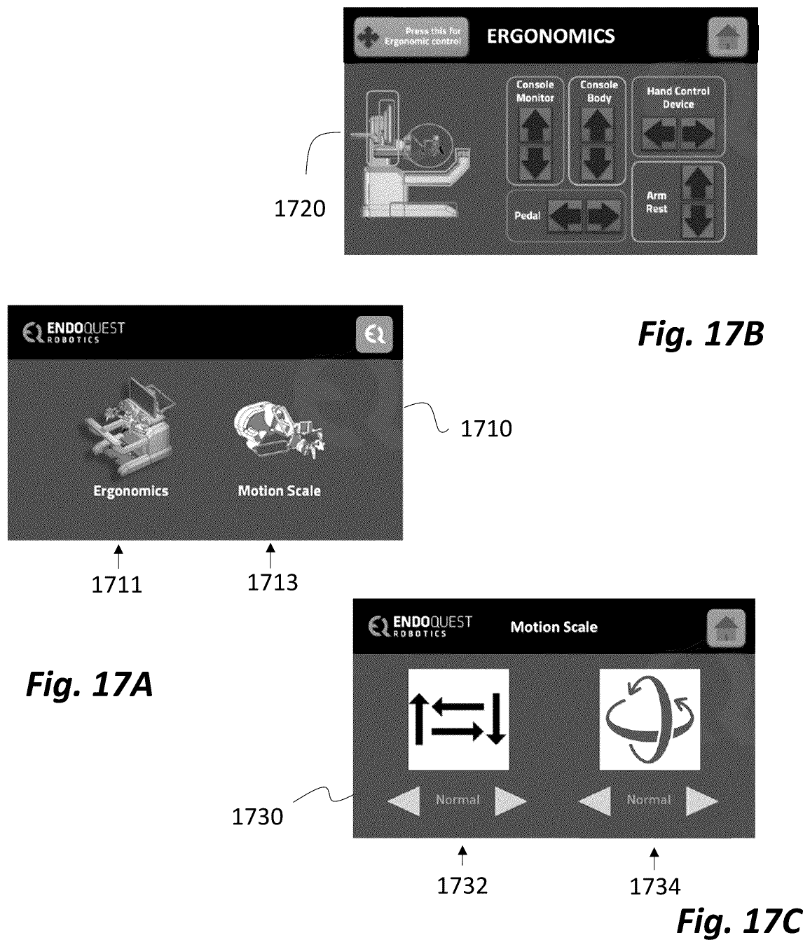

Exemplary graphical user interface screens 1710, 1720, 1730, for display on the touchscreen user interface 1541 of the physician console 1500 are illustrated respectively in FIGS. 17A, 17B and 17C. The placement of a small UI or GUI separately within easy reach of a physician simplifies and accelerates use without interfering with other UIs or GUIs, even momentarily. However, alternatively or additionally, these settings can be provided or integrated with a different UI or GUI elsewhere in connection with the system, such as a main system GUI.

An initial or home screen 1710 shown in FIG. 17A presents to an operator a selection of different functions in a central panel thereof. In the illustrated embodiment, the operator can select from an ergonomics settings screen icon 1711 and a motion scale settings screen icon 1713. A GUI controller, which can be independent or integrated into a higher-level controller, is programmed to respond to touch-selection of the icons and then to display the appropriate settings screen, such as those displayed in FIG. 17B and FIG. 17C, respectively.

An ergonomics settings screen 1720 (FIG. 17B) presents to an operator a set of graphical buttons to adjust height of the display or monitor 1510, the height of the console body 1520, including the horizontal position (distal/proximal) of the hand control devices 1530 from the operator, the horizontal position (distal/proximal) of the pedal tray 1550 from the operator, and the vertical position of the armrest 1540, as illustrated, as illustrated. To adjust a position of the respective component, the operator presses and holds the graphical button corresponding the desired movement until a satisfactory position is reached.

Additional adjustments optionally can be provided either as manual or motorized, including but not limited to, the lateral distance between hand control devices, the vertical height of the foot tray 1550, and horizontal position (distal/proximal) of the armrest 1540 from the operator, and the like. Moreover, further GUI screens can allow operators to save and retrieve a set position for one or more users, and/or preset settings based on the operator's height, enabling each operator to quickly access preferred settings, or allowing a new operator to quickly find a comfortable working position at the console 1500.

A motion scale settings screen 1730 (FIG. 17C) allows an operator to set preferred scaling of input movements from the hand control devices 1530 to the output movement of the instruments, including bending of joints, rotation of instruments, translation of instruments (such as surgical instruments, videoscope, and overtube 200). The motion scale setting screen 1730 includes translation scale settings 1732 and rotation scale settings 1734. Arrows displayed under the respective icon allow quick adjustments of global translation sensitivity or rotation sensitivity, with a current setting displayed between the arrows (e.g., Slow, Normal, Fast). Optionally, selection of the icon can lead an operator to a further menu where additional control settings for these aspects. Optionally, such settings can control scaling settings for individual translation and/or rotation axes. A home button can be displayed in this or other screens to return a user the home screen or other setting screen. Moreover, all of the foregoing GUI screens mentioned can have additional buttons or navigation panels above or below the main panels, or in other positions.

With certain illustrated embodiments of the systems, devices, methods and related user interfaces described above, it is to be appreciated that various non-limiting embodiments described herein may be used separately, combined, or selectively combined for specific applications. Further, some of the various features of the above non-limiting embodiments may be used without the corresponding use of other described features. The foregoing description should therefore be considered as merely illustrative of the principles, teachings, and exemplary embodiments of this invention, and not in limitation thereof.

Any module(s) disclosed herein can include any suitable hardware and/or software module(s) configured to perform any suitable function(s) (e.g., as disclosed herein, e.g., as described above). As will be appreciated by those skilled in the art, aspects of the present disclosure may be embodied as a system, method or computer program product. Accordingly, aspects of this disclosure may take the form of an entirely hardware embodiment, an entirely software embodiment (including firmware, resident software, micro-code, etc.), or an embodiment combining software and hardware aspects, all possibilities of which can be referred to herein as a “circuit,” “module,” or “controller.” A “circuit,” “module,” or “controller” can include one or more portions of one or more separate physical hardware and/or software components that can together perform the disclosed function of the “circuit,” “module,” or “controller”, or a “circuit,” “module,” or “controller” can be a single self-contained unit (e.g., of hardware and/or software). Furthermore, aspects of this disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of this disclosure may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of this disclosure may be described above with reference to flowchart illustrations and/or block diagrams of methods, apparatus (systems) and computer program products according to embodiments of this disclosure. It will be understood that each block of any flowchart illustrations and/or block diagrams, and combinations of blocks in any flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in any flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within 1% or within any other suitable percentage or number as appreciated by those having ordinary skill in the art (e.g., for known tolerance limits or error ranges).

The use of the term “substantially” in the Specification and Claims means largely but not wholly what is specified. The term “substantially” can also mean “consisting essentially of”.

With regard to degree, the term “substantially” in one aspect means greater than 50%, up to and including 100%. The term “substantially” in another aspect means 90% to 100%, inclusive. The term “substantially” in another aspect means 95% to 100%, inclusive. The term “substantially” in another aspect means 97% to 100%, inclusive. The term “substantially” in another aspect means 98% to 100%, inclusive. The term “substantially” in another aspect means 99% to 100%, inclusive. The term “substantially” in another aspect means 99.5% to 100%, inclusive. The term “substantially” in another aspect means 99.6% to 100%, inclusive. The term “substantially” in another aspect means 99.7% to 100%, inclusive. The term “substantially” in another aspect means 99.8% to 100%, inclusive. The term “substantially” in another aspect means 99.9% to 100%, inclusive.

With regard to function and corresponding functional language, the term “substantially” in the Specification and the Claims means sufficiently to such a degree of being precise such that performance of the prescribed action or task, from the perspective of one with ordinary skill in the art, is the same as though the object, element or step were exactly precise.

The term “predetermined” as used herein, including in the Specification and Claims, means an element, quantity or value, for example, which is selected in advance, where precise details, quantities or values can vary, but which nevertheless is relevant to the claimed invention.

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the systems, devices/apparatus and methods of the subject disclosure have been shown and described, those skilled in the art will readily appreciate that changes and/or modifications may be made thereto without departing from the scope of the subject disclosure. For example, those skilled in the art will readily appreciate that the various aspects of the invention described and illustrated throughout the specification, and components thereof, can be readily interchanged with one another and utilized alone or in any combination, without limitation, which is explicitly contemplated herein.

It is to be appreciated that the concepts, systems, circuits and techniques sought to be protected herein are not limited to use in the example applications described herein (e.g., industrial applications, medical/surgical applications), but rather may be useful in substantially any application where the subject devices, systems and methods find advantageous application. While particular embodiments and applications of the present disclosure have been illustrated and described, it is to be understood that embodiments of the disclosure not limited to the precise construction and compositions disclosed herein and that various modifications, changes, and variations can be apparent from the foregoing descriptions without departing from the spirit and scope of the disclosure as defined in the appended claims.

Accordingly, it is submitted that that scope of the patent should not be limited to the described embodiments but rather should be limited only by the spirit and scope of the following claims.

1. A user input device for a patient console of a robotic surgical system, comprising a user-selectable control for each of a plurality of motion devices of the patient console, the plurality of motion devices including:
a vertical lift device configured to provide up and down motion along a vertical axis;
a pitch rotation device configured to provide a pitch rotation about a pitch axis orthogonal to the vertical axis;
a translation device configured to provide sliding translation along a translation axis; and
a roll rotation device to rotate about a roll axis to provide a roll to an instrument controller assembly.

2. The user input device of claim 1, the plurality of motion devices further comprising:
a yaw rotation device configured to provide a yaw rotation about the vertical axis.

3. The user input device of claim 1, wherein an angle of the translation axis and the roll axis relative to horizontal is a function of the pitch rotation provided by the pitch rotation device.

4. The user input device of claim 1, wherein the user interface includes a digital display screen adapted and configured to display a graphic user interface, the graphic user interface configured to display graphic representations of buttons adapted and configured to control each of the plurality of motion devices.

5. The user input device of claim 1, wherein the user interface includes a button panel, including a button corresponding to each of the plurality of motion devices.

6. The user input device of claim 5, further comprising a button adapted and configured to selectively disengage operability of the button panel to inhibit unintentional activation of any of the plurality of motion devices.

7. The user input device of claim 1, wherein the user input device is arranged on a distal portion of the patient console, configured to be proximal to a patient.

8. The user input device of claim 1, wherein the user input device is arranged on a proximal portion of the patient console, configured to be distal to the patient.

9. A user input device for a patient console of a robotic surgical system, comprising a selectable control for each of a plurality of motion devices of the patient console, the plurality of motion devices including:
a vertical lift device configured to provide up and down motion along a vertical axis; and
a pitch rotation device supported by the vertical lift device and configured to provide a pitch rotation about a pitch axis orthogonal to the vertical axis.

10. The user input device of claim 9, the plurality of motion devices further comprising:
a translation device supported by the pitch rotation device and configured to provide sliding translation along a translation axis; and
a roll rotation device supported by the translation device to roll relative to the translation device about a roll axis to provide a roll to an instrument controller assembly,
wherein an angle of the translation axis and the roll axis relative to horizontal is a function of the pitch rotation provided by the pitch rotation device.

11. The user input device of claim 10, the plurality of motion devices further comprising:
a yaw rotation device interposed between the vertical lift device and the pitch device and configured to provide a yaw rotation about the vertical axis, and
wherein a direction of the translation axis and the roll axis is a function of the yaw rotation provided by the yaw rotation device.

12. A user interface for a robotic surgical system comprising:
a clutch button provided in connection with a robotic controller, the clutch button adapted and configured to selectively disengage at least one motion control device of the robotic controller to allow a user to manually manipulate a position of the robotic controller; and
a visual status indicator provided in connection with the robotic controller, adapted and configured to display an operational status of the robotic controller.

13. The user interface of claim 12, wherein the robotic controller is a robotic instrument controller or a robotic videoscope controller.

14. The user interface of claim 12, wherein the clutch button is provided on a distal end portion of the robotic controller.

15. The user interface of claim 12, wherein the at least one motion control device is a motorized actuator for effecting linear translation of the robotic controller along a longitudinal axis thereof.

16. The user interface of claim 12, wherein the visual status indicator includes a selectable color illumination source.

17. The user interface of claim 12, wherein the clutch button and the status indicator are both integrated into a single illuminated button.
